(19) United States
(12) Patent Application Publication
(10) Pub. No.: US 2009/0216302 A1
(43) Pub. Date: Aug. 27, 2009

(54) Irradition apparatus for irradiating a human body

(76) Inventor: Ernst Smolka, Goldbach (DE)

Correspondence Address: WESTMAN CHAMPLIN & KELLY, P.A., SUITE 1400, 900 SECOND AVENUE SOUTH, MINNEAPOLIS, MN, 55402, US

(21) Appl. No.: 12/043,393

(22) Filed: Mar. 6, 2008

(30) Foreign Application Priority Data
Mar. 7, 2007 (EP) 07103693.3

(51) Int. Cl. A61N 5/06

(52) U.S. Cl. 607/94

(57) ABSTRACT

The subject matter of the present invention relates to an irradiation apparatus for irradiating a human body, with at least two types of tubular low-pressure lamps which emit UVA and/or UVB radiation and which are alternatingly disposed so as to lie parallel and close to one another, covering at least part of a radiation field, with the low-pressure lamps of the second type B emitting a higher UVB radiation than the low-pressure lamps of the first type A, with the diameter of the low-pressure lamps of the second type B being smaller than the diameter of the low-pressure lamps of the first type A, i.e., with the diameter ratio being approximately 5:12, with the low-pressure lamps of at least the second type B, preferably also the low-pressure lamps of the first type A, having a length of at least 120 cm, preferably at least 180 cm, and with the low-pressure lamps of the first type A and the low-pressure lamps of the second type B being separately activated and with their erythemal power also being separately set, and with the low-pressure lamps of the second type B being pure UVB lamps.

### FIELD OF THE INVENTION

The subject matter of the present invention relates to an irradiation apparatus for irradiating a human body, in particular for whole-body irradiation, with at least two types of tubular low-pressure lamps which emit UVA and/or UVB radiation, as well as a method of operating an irradiation apparatus for whole-body irradiation, especially of a human body, with at least two types of tubular low-pressure lamps which emit UVA and/or UVB radiation.

### BACKGROUND OF THE INVENTION

Conventional irradiation apparatuses, in particular tanning lamps, are divided into various irradiation classes, with a main classification distinguishing between a first group of irradiation apparatuses with focus on UVA radiation and a second group of apparatuses with focus on UVB radiation.

The UVA range of ultraviolet radiation is defined by a wavelength from 315 to 400 nm. The shorter-wave range of the ultraviolet UVB radiation is in a range from 280 to 315 nm.

Given the conventional distinction of irradiation apparatuses into different classes, the manufacturers of irradiation apparatuses must produce a large number of different apparatuses, which leads to time- and cost-consuming production processes, time- and cost-consuming inventory management, etc. The operators of tanning salons also must have a large number of different types of irradiation apparatuses on hand so as to be able to meet the specific requirements demanded in a specific case of application.

The reason is that a radiation spectrum of a specific low-pressure lamp is obtained by mixing several fluorescent materials. On excitation, which is conventionally performed with Hg radiation, these fluorescent materials emit radiation of different wavelengths. The need for different spectra with a specifiable ratio between UVA radiation and UVB radiation is high. This has led to many types of lamps, which, as already mentioned earlier, entails high costs for the manufacturing process, inventory management, and logistics.

Therefore, it is not surprising that this problem has already been tackled in the prior art which has proposed irradiation apparatuses in which the radiated power can be set as to its UVA/UBV ratio. One example of such an irradiation apparatus is disclosed in DE 78 26 003 U1.

In this prior-art case, a plurality of low-pressure lamps of a first type A as well as low-pressure lamps of a second type B are disposed in a radiation field, with the low-pressure lamps of the second type B emitting a higher UVB radiation than the low-pressure lamps of the first type A. The concrete solution proposed in this document provides that one low-pressure lamp of the second type B each be disposed between groups of two or three low-pressure lamps of the first type A. The distances between the low-pressure lamps are comparatively large. In addition, the diameters of the low-pressure lamps of the first type A are identical to the diameters of the low-pressure lamps of the second type B.

### SUMMARY OF THE INVENTION

Thus, the problem to be solved by the present invention is to make available an irradiation apparatus as well as an associated method in which both UVA and UVB radiation can be emitted and in which the ratio of UVA and UVB radiation can be set while ensuring the greatest possible uniform surface distribution of the radiated power.

This problem is solved with an irradiation apparatus according to the features of claim 1 and with a method according to the features of claim 16.

Proposed is an irradiation apparatus for irradiating a human body, with at least two types of tubular low-pressure lamps which emit UVA and/or UVB radiation and which are alternately disposed so as to lie parallel and close to one another, covering at least part of a radiation field, with the low-pressure lamps of the second type B emitting a higher UVB radiation than the low-pressure lamps of the first type A, with the diameter of the low-pressure lamps of the second type B being smaller than the diameter of the low-pressure lamps of the first type A; i.e., with the diameter ratio being approximately 5:12, with the low-pressure lamps of at least the second type B, preferably also the low-pressure lamps of the first type A, having a length of at least 180 cm, and with the possibility of separately controlling the low-pressure lamps of the first type A and the low-pressure lamps of the second type B as well as separately setting the erythemal power of said lamps.

The configuration alternating between low-pressure lamps of the first type A and low-pressure lamps of the second type B as proposed by the present invention can be used either across the entire radiation field of an irradiation apparatus or only across parts of the irradiation apparatus, e.g., only in the center and/or with a gap for incorporating a face tanning apparatus.

Useful further developments follow from the dependent claims.

Thus, the invention proposes an irradiation apparatus which markedly differs in several aspects from the prior art described above. First, the low-pressure lamps of the first type A and the low-pressure lamps of the second type B are alternatingly disposed and in particular, the low-pressure lamps of the second type B are designed to have a smaller diameter so that the homogeneity and UV power of the radiation emitted by the low-pressure lamps of the first type A is interfered with as little as possible and the spacing between the low-pressure lamps of the first type A is as close as possible.

Uniform irradiation both in the UVA and in the UVB range was not possible with an irradiation apparatus of the design proposed in the prior art discussed above. In contrast, the applicant realized that the low-pressure lamps of the first type A should be disposed as uniformly as possible in the radiation field and that this uniformity is considerably disrupted, even if only one low-pressure lamp with a different radiation spectrum with an identical diameter is added, so that according to the present invention, low-pressure lamps with a smaller diameter are proposed.

Presently, tanning salons are often equipped with T12 low-pressure lamps which have a diameter of 12/8 of an inch=38.1 mm. If individual T12 low-pressure lamps of the first type A were to be replaced with low-pressure lamps of the second type B, the spacing between the low-pressure lamps of the first type A would be too large. Based on the reasoning of the present invention, the low-pressure lamps predominantly responsible for UVB radiation are therefore designed so as to have a smaller diameter. When T12 low-pressure lamps are used for type A, T5 low-pressure lamps are used [sic; for type B] according to the present invention. The low-pressure lamps of the second type B, in particular the T5 low-pressure lamps, have a length of at least 120 cm, preferably at least 180 cm, so that the T5 low-pressure lamps extend across the entire length of the radiation field, thus ensuring that to achieve the required overall length of 180 cm, it is not necessary to dispose two shorter dimensioned low-pressure lamps one after the other, which would entail a more time- and cost-intensive assembly, since a larger inventory of lamps must be on hand and the installation and activation are more complicated.

To further improve a uniform radiation distribution, the low-pressure lamps of the first type A and the low-pressure lamps of the second type B should be closely disposed next to one another, in particular the spacing should not be greater than one quarter of the lamp diameter of the low-pressure lamps of the first type A and preferably not greater than one quarter of the lamp diameter of the low-pressure lamps of the second type B.

According to one aspect of the present invention, the low-pressure lamps of the second type B, in particular the T5 low-pressure lamps, are pure UVB lamps.

In the context of the present invention, “pure” UVB lamps are defined as lamps for which the erythemal power in the UVB range (280 to 315 nm) is nearly 100%, in particular more than 98.5%, for a first concrete fluorescent coating 99.6% and for an alternative fluorescent coating 99.8%.

The low-pressure lamps of the second type B are preferably pure UVB lamps.

According to a preferred embodiment, the low-pressure lamps of the first type are pure UVA lamps. In the context of the present invention, “pure” UVA lamps are defined as lamps with a UVB/UVA ratio of <0.25% (UVA: 315 to 400 nm).

In a concrete embodiment of the low-pressure lamps of the first type A, which were concretely designed as T12 lamps, an erythemal power of 0.273 μW/cm² resulted for the range from 250 to 320 nm, and an erythemal power of 0.532 μW/cm² for the range from 321 to 400 nm.

In a concrete embodiment of a low-pressure lamp of type B, which was concretely designed as a T5 lamp, an erythemal power of 18.26 μW/cm² resulted for the range from 250 to 320 nm, and an erythemal power of 0.078 μW/cm² for the range from 321 to 400 nm.

The erythemal power was in all cases determined pursuant to Cie. Puppl. [sic] 17.4.

According to another preferred aspect of the present invention, the configuration of the low-pressure lamps of the first type A and of the low-pressure lamps of the second type B ensures that the radiation field in projection toward the main direction of radiation is in essence completely covered by UV light sources in the form of low-pressure lamps. Using this measure of essentially covering the entire radiation field, in association with the basic idea of the present invention, which is the configuration in which low-pressure lamps of type A alternate with low-pressure lamps of type B, with the diameter of the low-pressure lamp of type B being reduced, an especially uniform radiation distribution is achieved.

According to a preferred aspect of the present invention, the low-pressure lamps of the second type B are slightly staggered with respect to the low-pressure lamps of the first type A in the direction of the main direction of radiation, preferably in such a manner that they terminate at least for the most part flush along the side facing the main direction of radiation.

Using this measure, the low-pressure lamps of the first type A and the low-pressure lamps of the second type B can be positioned even more closely next to one another, and the smaller diameter of the low-pressure lamp of the second type B at the same time ensures the shorter distance of the projection toward the direction of radiation, on the one hand, and in an essentially flush line along their side facing the main radiation, on the other hand.

As an alternative, it is, however, also possible to stagger the low-pressure lamps of the second type B with respect to the low-pressure lamps of the first type A slightly in the direction facing away from the main direction of radiation, preferably in such a manner that they terminate essentially flush along the side facing away from the main direction of radiation. Again, this leads to advantages of a closer spacing between each other, on the one hand, and to an essentially flush termination along the side facing away from the main direction of radiation.

The low-pressure lamps of the first type A can have a length of at least 60 cm, preferably of at least 150 cm, more preferably of at least 180 cm, and most preferably of at least 200 cm. Especially when implementing an irradiation apparatus for whole-body irradiation, lengths of at least 180 cm or 6 feet or of at least 200 cm can be useful so that low-pressure lamps can be disposed along the entire length of the radiation field.

The low-pressure lamps of the second type B have a length of at least 120 cm, preferably of at least 180 cm or 6 feet, possibly even of at least 200 cm. This ensures in a similar manner that the low-pressure lamps can extend along the entire length of the radiation field (with or without an integrated face tanning apparatus) and that it is not necessary, for example, to have two low-pressure lamps be aligned one after the other. The configuration of low-pressure lamps of the second type in the form of T5 lamps with lengths of at least 180 cm, in particular of at least 200 cm, is an independent aspect of the present invention which as such is also claimed independently.

In a preferred embodiment, approximately 15% to 35%, preferably approximately 30%, of the radiation field are covered by low-pressure lamps of the second type B.

According to another preferred embodiment, approximately 65% to 75%, preferably approximately 70% of the radiation field is covered by low-pressure lamps of the first type A.

As already mentioned above, a low-pressure lamp with a diameter of ⅝ of an inch of the type of a T5 tube is also independently claimed, in particular for use in an irradiation apparatus of the type explained above, which irradiation apparatus is especially characterized in that it has a ratio between length and diameter of 114 to 130, preferably of approximately 125.

In addition, it is possible to integrate a face tanning apparatus into the radiation field, which face tanning apparatus comprises, for example, a plurality of short T5 lamps or high-pressure quartz lamps. The face tanning apparatus and its lamps can also be activated separately from the remaining low-pressure lamps of the radiation field and be adjusted to the irradiance desired.

The irradiation apparatus according to the present invention can be used especially for whole-body irradiation and/or for the cosmetic tanning of a human body.

Depending on the specific use of the irradiation apparatus proposed by the present invention, special fluorescent materials can be used for the low-pressure lamps of the first type A and the low-pressure lamps for the second type B. The same applies to glasses for the transmission and/or potentially used filters. The irradiation apparatus according to the present invention can also be used for light therapy.

In addition, the present invention also proposes a method of operating an irradiation apparatus, in particular for irradiation of a human body by means of at least two types of tubular low-pressure lamps which emit UVA and/or UVB radiation and which are disposed parallel and in close vicinity to one another, covering at least part of a radiation field, with the low-pressure lamps of the second type B emitting a higher UVB radiation than the low-pressure lamps of the first type A, with the area fraction covered in the radiation field by low-pressure lamps of the first type A being at least twice as large as the area fraction covered by low-pressure lamps of the second type B, with the low-pressure lamps of the second type B having a diameter that is reduced in comparison to the diameter of the low-pressure lamps of the first type A, with the low-pressure lamps of at least the second type B, preferably also the low-pressure lamps of the first type A, having a length of at least 120 cm, preferably at least 180 cm, and with the low-pressure lamps of the first type A and the low-pressure lamps of the second type B being separately activated and with their erythemal power also being separately set.

According to another preferred aspect of the present method, the UVB power is set by increasing or reducing the power of the low-pressure lamps of the second type B with respect to a preferably constant power of the low-pressure lamps of the first type A.

The power of the low-pressure lamps of the first type A and/or of the low-pressure lamps of the second type B can be set, in particular restricted, via ballast devices associated with each of the low-pressure lamps.

According to another preferred aspect of the present invention, the UVA power of the low-pressure lamps of the first type A and/or the UVB power of the low-pressure lamps of the second type B are set as a function of the skin type of a person to be exposed to radiation.

The skin type of a person to be exposed to radiation can also be determined by means of special measuring devices. Based on the results of such a measurement, the irradiation apparatus can subsequently be set and the method of operating the irradiation apparatus can be determined for the specific case at hand.

According to yet another preferred aspect of the present invention, the skin type of a person to be exposed to radiation is automatically determined and is especially preferably automatically transmitted to the irradiation apparatus and/or automatically used to select the parameters of the method according to the present invention. In particular, it is possible for a measuring device for determining the skin type of a person to be exposed to radiation to be integrated in the irradiation apparatus.

The two considerations mentioned last allow an especially efficient use of the irradiation apparatus proposed by the present invention since the irradiation can now be carried out with a single irradiation apparatus, in each case taking into account the special requirements.

Using the proposed irradiation apparatus and the proposed method, it is possible to individually set the desired irradiation spectrum in UVA and UVB from only UVA, any mixture of A and B, to only UVB. In addition, the irradiance of A and B can be adjusted by way of the lamp wattage.

As already discussed in the introduction, given the conventionally used distinction of irradiation apparatuses into different classes, the manufacturers must produce a large number of different apparatuses, which leads to time- and cost-consuming production processes, time- and cost-consuming inventory management, etc. The operators of tanning salons also must have a large number of different types of irradiation apparatuses on hand so as to be able to meet the specific requirements demanded in a specific case of application. The irradiation apparatus according to the present invention and the method according to the present invention makes it possible to implement this with simple software in a single apparatus.

The present invention, including additional features and advantages, will be explained in greater detail below based on a description of practical examples and with reference to the appended drawings.

### BRIEF DESCRIPTION OF THE DRAWINGS

As can be seen:

FIG. 1 shows an irradiation apparatus known from the prior art;

FIG. 2 shows an irradiation apparatus according to an embodiment of the present invention in which T5 low-pressure lamps with a predetermined UVB percentage are disposed between T12 low-pressure lamps with a UVA spectrum;

FIG. 3 shows an alternative embodiment an of irradiation apparatus in which T12 low-pressure lamps and T5 low-pressure lamps are alternately disposed, with the T12 low-pressure lamps having a UVA spectrum and the T5 low-pressure lamps having a predetermined UVB percentage;

FIG. 4 shows a first embodiment of a configuration of the low-pressure lamps in a radiation field, and

FIG. 5 shows a second diagrammatic sketch which explains an alternative configuration of the low-pressure lamps in a radiation field.

### DETAILED DESCRIPTION OF ILLUSTRATIVE EMBODIMENTS

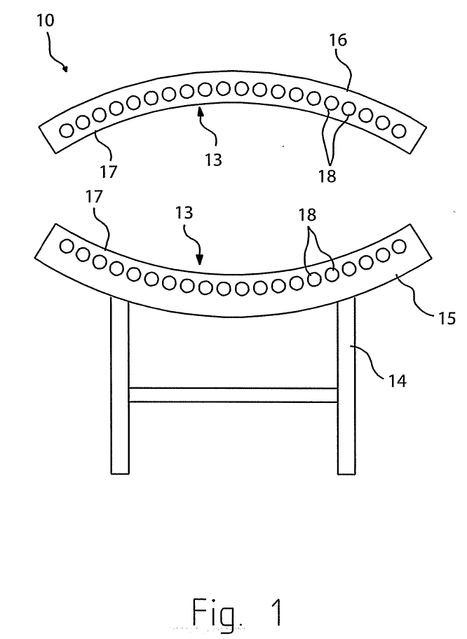

FIG. 1 shows a diagrammatic sketch of an irradiation apparatus 10 known from the prior art. This prior-art irradiation apparatus 10 comprises two housings 15, 16 which, on the sides facing each other, have one UV-permeable pane 17 each. In the assembled position, the lower housing 15 defines a bed and can be mounted on a frame-like support structure 14. In the assembled position, the upper housing 15 defines a so-called “sky” and can, for example, be attached to the lower housing 15 by means of a hinge joint. As an alternative, the upper housing 16 might be mounted differently, for example, directly on a ceiling of a room.

Both in the lower housing 15 and in the upper housing 16, a plurality of low-pressure lamps 18 are conventionally disposed parallel to one another, each defining a radiation field 13. The main direction of radiation of the radiation field 13 in the lower housing 15, which radiation field is defined by the low-pressure lamps 18, is vertically oriented upwardly in the direction of the upper housing 16. Similarly, the main direction of radiation of the low-pressure lamps 18 that define the radiation field 13 of the upper housing 16 is vertically oriented downwardly in the direction of the lower housing 15.

The prior-art irradiation apparatuses 10 also attempt to achieve the most homogeneous possible radiation characteristics of the radiation fields 13 so as to make it possible to expose a human body as uniformly as possible to radiation. At the same time, however, it was desirable to design irradiation apparatuses in which the ratio of the radiated power in the UVA and UVB range can be adjusted. The irradiation apparatuses with an adjustable ratio between UVB and UVA power proposed by the prior art, however, do not meet the requirements of a relatively homogeneous radiation demanded by a specific application.

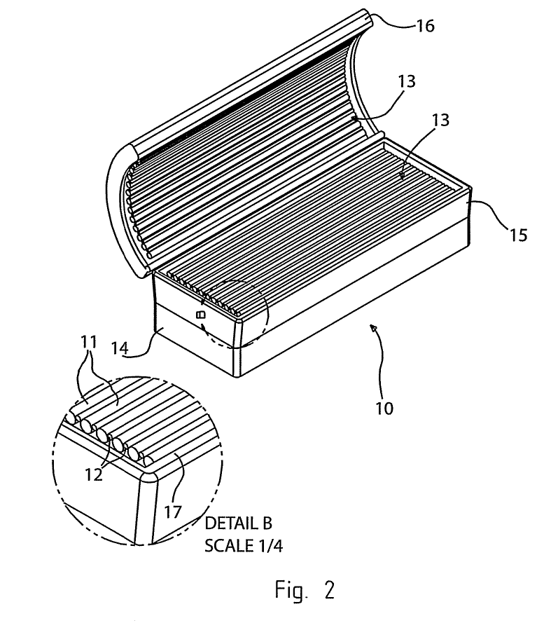

In FIG. 2, an embodiment of an irradiation apparatus 10 according to the present invention is illustrated. An upper housing 16 is swivelably hinged to a lower housing 15. The lower housing 15 is mounted on a frame-like support structure 14. Both in the lower housing 15 and in the upper housing 16, a plurality of low-pressure lamps 11 of a first type A as well as a plurality of low-pressure lamps 12 of a second type B are alternatingly disposed in a parallel alignment with respect to one another (see also detail drawing A).

In this specific embodiment shown, the low-pressure lamps of the first type A are T12 low-pressure lamps and T5 low-pressure UV fluorescent lamps and designed to emit a UVA spectrum. T12 low-pressure UV fluorescent lamps are known from the prior art and have a diameter of 12/8 of an inch=38.1 mm. Such comparatively thick low-pressure lamps are highly suitable to cover a radiation field 13 since a wide area can be covered with comparatively low-cost lamps.

The radiation field 13 within the lower housing 15 can have a length, for example, of 200 cm and a width of 70 cm. The radiation field 13 of the upper housing 16 has a corresponding length and, because of the curved design, an arc length of slightly more than 70 cm. Within the radiation field 13 of the upper housing 16, an additional area for a face tanning field can be disposed on the edge (on one head end), which face tanning field can be formed, for example, by a plurality of high-pressure UV lamps or T5 lamps.

In addition, a face tanning apparatus which is formed by short T5 low-pressure lamps or high-pressure quartz lamps can be integrated into the radiation field 13. These lamps of the face tanning apparatus can be activated separately from the lamps in the main field and be set to a desired irradiance.

In the embodiment illustrated in this figure, however, additional low-pressure lamps 12 of the second type B which in this case are T5 low-pressure UV fluorescent lamps are disposed between the T12 low-pressure lamps 11 of the first type A. The low-pressure lamps 12 of the second type B which in this case are T5 lamps also emit an erythemal UVB percentage. T5 low-pressure UV fluorescent lamps are also known from the prior art, although only in comparatively short lengths. T5 low-pressure UV fluorescent lamps have a diameter of ⅝ of an inch, corresponding to approximately 15.9 mm. As can be particularly well seen in detail drawing A, in the direction of radiation, the low-pressure lamps 12 of the second type B are staggered with respect to the low-pressure lamps 11 of the first type A in such a manner that they terminate essentially flush along their side facing the upper half of the housing 16 and along their side facing the direction of radiation. The same solution is provided in the radiation field 13 within the upper housing 16. In the direction of radiation, the low-pressure lamps 12 of the second type B are staggered with respect to the low-pressure lamps 11 of the first type A so that they essentially terminate flush along their side facing the lower housing 15 and along their side facing the direction of radiation.

The low-pressure lamps 11 of the first type A and/or the low-pressure lamps 12 of the second type B can comprise a reflector which is integrated into their bulbs and which can be an Al₂O₃ coating. The Al₂O₃ coating can be implemented, for example, by applying two coatings to approximately half the circumference of the inside of the bulbs of the low-pressure lamps 11 of the first type A and/or of the low-pressure lamps 12 of the second type B. As an alternative or in addition thereto, a reflector can also be disposed in the housing 15,16 behind the low-pressure lamps 11,12, i.e., on the side facing away from the direction of radiation. These reflectors which are integrated in housing 15,16 can also have a metal base, for example, an aluminum base.

Relative to their overall erythemal UV power, the low-pressure lamps 12 of the second type B can typically emit approximately 5% to nearly 100% of UVB power.

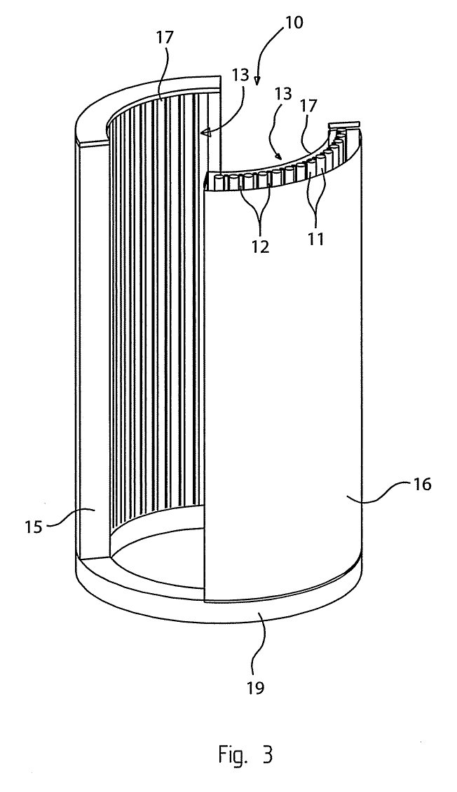

FIG. 3 shows another modified embodiment of an irradiation apparatus. In the embodiment illustrated in this figure, two vertical housings 15′,16′ are mounted on a ring-shaped base element 19. On their sides facing each other, the vertical housings 15′,16′ again have a UV-permeable pane 17. Disposed on one side between the vertical housings 15′,16′ is an opening so that a user can enter from the side and can be exposed to UV light emitting from the vertical housings 15,16 [sic; 15′,16′] while standing upright approximately in the center of the ring-shaped base element 19.

To this end, the vertical housings 15′,16′ also comprise a plurality of low-pressure lamps 11 of the first type A as well as a plurality of low-pressure lamps 12 of the second type B which are disposed alternatingly in parallel alignment and covered by the UV-permeable pane 17. In this case again, the low-pressure lamps 12 of the second type B can be staggered in the direction of radiation (i.e., in the direction of the respective oppositely disposed vertical housing 16′,15′) with respect to the low-pressure lamps 11 of the first type A in such a manner that they terminate essentially flush along their side facing the direction of radiation or in the direction of the respective oppositely disposed vertical housing 16′,15′.

In a manner similar to that shown in the embodiment of FIG. 2, the low-pressure lamps 11,12 can again have an integrated reflector, for example, an Al₂O₃ coating, and/or a reflector can be integrated behind the low-pressure lamps 11,12 in the vertical housing 15′,16′.

Both in the embodiment seen in FIG. 2 and in the embodiment seen in FIG. 3, the low-pressure lamps 11,12 can have a length of up to two meters or more. T5 low-pressure UV fluorescent lamps with a length of 180 cm or 6 ft or more are so far not known in the prior art, so that such a light source, in particular for emitting UV light and/or specifically for use in UV irradiation apparatuses, is also independently claimed.

Within the scope of the present invention, it is, of course, also possible to use low-pressure lamps 11 of the first type A or low-pressure lamps 12 of the second type B which do not cover the entire radiation field 13. Especially with low-pressure lamps 12 of the second type B in the form of T5 low-pressure UV fluorescent lamps, it is possible to position prior-art T5 tubes that are 4 ft and 2 ft long one behind the other so as to obtain a length of 6 ft.

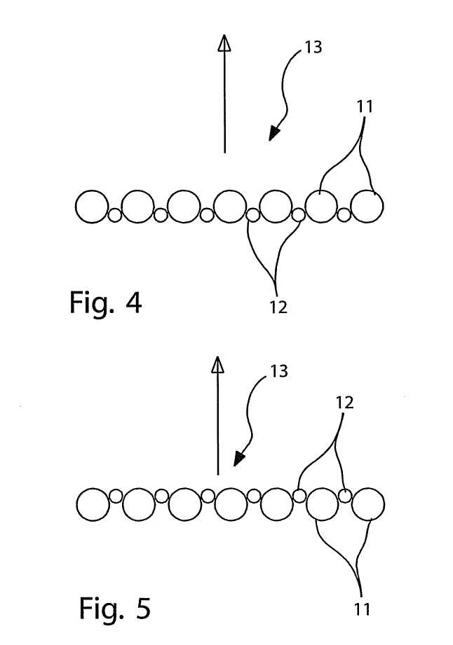

In FIGS. 4 and 5, two potential configurations of the low-pressure lamps 12 of the second type B relative to the low-pressure lamps 11 of the first type A are shown. In the diagrammatic sketch in FIG. 4, the low-pressure lamps 12 of the first type B, in the direction opposite to the direction of radiation, are staggered to the rear with respect to the low-pressure lamps 11 of the first type A in such a manner that they essentially terminate flush along their side facing away from the direction of radiation.

As seen in FIG. 4, this leads to a radiation field 13 in which, in projection toward the direction of radiation, practically no gaps are formed between the low-pressure lamps 11 and 12, so that at least for the most part, the entire area of the radiation field 13 is covered.

With the configuration shown in FIG. 5, an essentially full-area coverage of the radiation field 13 by low-pressure lamps 11 or 12 is possible as well, with the low-pressure lamps 12 of the second type B in this case being staggered with respect to the low-pressure lamps 11 of the first type A in the direction of the radiation, as already mentioned in the explanation of the embodiments seen in FIGS. 2 and 3, i.e., in this specific case in such a manner that they terminate essentially flush along their side facing the direction of radiation.

### LIST OF REFERENCE NUMERALS

10 Irradiation apparatus
11 Low-pressure lamps (type A)
12 Low-pressure lamps (type B)
13 Radiation field
14 Frame-like support structure
15,16 Housing
15′,16′ Vertical housing
17 UV-permeable pane
18 Low-pressure lamps (from the prior art)
19 Ring-shaped base element

1. An irradiation apparatus for irradiating a human body with at least two types of tubular low-pressure lamps which emit UVA and/or UVB radiation, which lamps are alternatingly disposed so as to lie parallel and close to one another, covering at least part of a radiation field, with the low-pressure lamps of the second type B emitting a higher UVB radiation than the low-pressure lamps of the first type A, with the diameter of the low-pressure lamps of the second type B being smaller than the diameter of the low-pressure lamps of the first type A, i.e., with the diameter ratio being approximately 5:12, with the low-pressure lamps of at least the second type B, preferably also the low-pressure lamps of the first type A, having a length of ≧120 cm, preferably of ≧180 cm, and with the possibility of separately controlling the low-pressure lamps of the first type A and the low-pressure lamps of the second type B as well as separately setting the erythemal power of said lamps.

2. The irradiation apparatus as in claim 1, characterized in that the low-pressure lamps of the second type B are pure UVB lamps.

3. The irradiation apparatus as in claim 1, characterized in that the low-pressure lamps of the first type (A) are pure UVA lamps.

4. The irradiation apparatus as in claim 1, characterized in that the low-pressure lamps of the first type A are formed by T12 tubes, i.e., low-pressure lamps with a diameter of 12/8 of an inch=38.1 mm.

5. The irradiation apparatus as in claim 1, characterized in that the low-pressure lamps of the second B are formed by T5 tubes, i.e., low-pressure lamps with a diameter of ⅝ of an inch, corresponding to 15.9 mm.

6. The irradiation apparatus as in claim 1, characterized in that in the radiation field, the low-pressure lamps of the first type A and the low-pressure lamps of the second type B are lying close to one another so that they are spaced no more than one quarter of the lamp diameter of the low-pressure lamps of the first type A from one another, preferably no more than one quarter of the lamp diameter of the low-pressure lamps of the second type B from one another.

7. The irradiation apparatus as in claim 1, characterized in that in projection toward the main direction of radiation, essentially the entire area of the radiation field, or at least 95% of it, is covered alternately by low-pressure lamps of the first type A and by low-pressure lamps of the second type B.

8. The irradiation apparatus as in claim 1, characterized in that in the direction of the main direction of radiation, the low-pressure lamps of the second type B are slightly staggered with respect to the low-pressure lamps of the first type A so that they terminate essentially flush along their side facing the main direction of radiation.

9. The irradiation apparatus as in claim 1, characterized in that the low-pressure lamps of the second type B are slightly staggered with respect to the low-pressure lamps of the first type A in the direction opposite to the main direction of radiation in such a manner that they terminate at least essentially flush with each other on their side that faces away from the main direction of radiation.

10. The irradiation apparatus as in claim 1, characterized in that the low-pressure lamps of the first type A have a length of at least 60 cm, preferably of at least 150 cm, more preferably of at least ≧180 cm, and most preferably of at least 200 cm.

11. The irradiation apparatus as in claim 1, characterized in that approximately 15% to 35%, preferably approximately 30%, of the radiation field are covered by low-pressure lamps of the second type B.

12. The irradiation apparatus as in claim 1, characterized in that approximately 65% to 75%, preferably approximately 70%, of the radiation field is covered by low-pressure lamps of the first type A.

13. The irradiation apparatus as in claim 1, characterized in that a face tanning apparatus is integrated into the radiation field, which face tanning apparatus comprises a plurality of T5 low-pressure lamps or a plurality of high-pressure quartz lamps, with the possibility of controlling the face tanning apparatus and/or its lamps separately from the low-pressure lamps of the remaining radiation field and of adjusting it to the irradiance desired.

14. A low-pressure lamp with a diameter of ⅝ of an inch in the form of a T5 tube, in particular for use in an irradiation apparatus as in claim 1, characterized in that the low-pressure lamp has a ratio between length and diameter of 114 to 130, preferably of approximately 125.

15. The use of an irradiation apparatus as in claim 1 for cosmetically tanning a human body.

16. A method of operating an irradiation apparatus, in particular of exposing a human body to radiation, with at least two types of tubular low-pressure lamps which emit UVA and/or UVB radiation, which lamps are disposed so as to lie parallel and close to one another and cover a part of a radiation field, with the low-pressure lamps of the second type B emitting a higher UVB radiation than the low-pressure lamps of the first type A, with the area fraction of the radiation field covered by low-pressure lamps of the first type A being at least twice as large as the area fraction covered by low-pressure lamps of the second type B, with the low-pressure lamps of the second type B having a diameter that is smaller than the diameter of the low-pressure lamps of the first type A, with the low-pressure lamps of at least the second type B, preferably also the low-pressure lamps of the first type A, having a length of ≧120 cm, preferably a length of ≧180 cm, and with the low-pressure lamps of the first type A and the low-pressure lamps of the second type B being separately activated and with the erythemal power of said lamps also being separately set.

17. The method as in claim 16, characterized in that to set the UVB power, the power of the low-pressure lamps of the second type B is increased and/or reduced with respect to a preferably constant power of the low-pressure lamps of the first type A.

18. The method as in claim 17, characterized in that the UVB power of the low-pressure lamps of the second type B is adjusted as a function of the skin type of a person to be exposed to the radiation.

19. The method as in claim 18, characterized in that the skin type of a person to be exposed to the radiation is automatically determined.
